(19) United States
(12) Patent Application Publication
(10) Pub. No.: US 2013/0123648 A1
(43) Pub. Date: May 16, 2013

(54) MEDICAL DIAGNOSIS AND TREATMENT USING MULTI-CORE OPTICAL FIBERS

(71) Applicants: LEONTIOS STAMPOULIDIS, ATHENS (GR); EFSTRATIOS KEHAYAS, ATHENS (GR)

(72) Inventors: LEONTIOS STAMPOULIDIS, ATHENS (GR); EFSTRATIOS KEHAYAS, ATHENS (GR)

(21) Appl. No.: 13/674,587

(22) Filed: Nov. 12, 2012

Related U.S. Application Data
(60) Provisional application No. 61/558,594, filed on Nov. 11, 2011.

(51) Int. Cl. A61B 6/00; A61N 5/06

(52) U.S. Cl. 600/478

(57) ABSTRACT

Devices and techniques are disclosed for delivering light from a plurality of single emitter lasers to a biological tissue and detecting light from a biological tissue with a plurality of detector components using multi-core optical delivery and detection fiber or fibers for minimally invasive treating and/or diagnosing conditions and/or diseases in an individual.

### RELATED APPLICATIONS

This application claims the benefit of U.S. Provisional Application No. 61/558,594, filed on Nov. 11, 2011, commonly owned and assigned to the same assignee hereof.

### BACKGROUND

### Field

The present disclosure relates to therapeutic and diagnostic techniques that engage light emission and detection.

### Background

Minimally-invasive procedures involving interaction between light and tissue are rapidly gaining momentum due to the progress in fiber optic technology and the corresponding inherent advantages compared to traditional invasive operations. For example, Photodynamic therapy (PDT) is a clinical treatment modality that combines (i) the administration of a photosensitising drug (often called a photosensitizer), (ii) irradiation by visible light at the appropriate wavelength and (iii) molecular oxygen formation, in order to cause destruction of selected cells.

The photosensitiser, when introduced into the body, accumulates in rapidly dividing cells, such as cancerous tumors which are particularly vascular type tissue. A measured light dose is then able to be applied (irradiated) onto the target tissue.

Light irradiation activates the photosensitizer through a series of electronic excitations and elicits a series of cytotoxic reactions, which are primarily dependent on, but can be independent of, the generation of reactive oxygen species.

In the case of cancer treatment, the destruction of tumours by PDT is achieved by the contribution of three main processes: (i) direct cellular damage, (ii) permanent or temporary damage to tumour vasculature, the later exacerbated by reperfusion injury and (iii) by activation of the immune response against tumour cells.

PDT is applied extensively for interstitial diagnosis and treatment due to the easy direction of laser beams through optical fibers into and outside the body. A typical PDT system consists of a laser coupled to an optical delivery fiber or multiple optical delivery fibers that deliver the light to a biological tissue. A portion of the delivered light is scattered by the tissue. Then the PDT system includes an additional optical monitoring fiber or multiple optical monitoring fibers that collect the scattered light and deliver it to detection components for diagnostic or treatment monitoring purposes.

The treatment efficiency and treatment duration depend on the energy and spatial coherence of the light delivered through the delivery fiber to the biological tissue to be treated. In PDT the necessary optical power required to be delivered through an optical fiber is typically in the Watt-level.

Typically Broad Area Lasers (BAL) are used to deliver such high power. BALs are lasers where the emitting region at the front facet has the shape of a broad stripe. Although BALs are compact and can provide optical powers in excess of 1W through optical fibers, the necessity to have a broad stripe at the emitting side has two major drawbacks. Firstly they require a larger fiber core (greater than 400 um) for efficient coupling and secondly the spatial coherence is low, compared to that of a Single Emitter Laser (SEL).

Two methods have been used to deliver high light energy to biological tissues for PDT. The first method involves BALs, which—due to their poor spatial coherence—require thick optical fibers with typical core diameter of 400 um or more to deliver the treatment light. The second method involves external feedback mechanisms to change the beam coherence properties of BALs and enable coupling to thinner optical fibers, however in this case part of the optical power is lost.

Of recent, fiber-optic technology is also being considered for a variety of medical uses, including for minimally-invasive techniques and other techniques where more accurate control of light illumination of therapeutic light is important. Applications, other than PDT (where light is used to activate drugs), include treatments for, for example, vein treatment and angioplasty, where efficient and accurate control of the illuminated light pattern, direction and intensity is critical.

FIGS. 1 and 2 illustrate two different conventional PDT systems. First configuration 110 includes laser 112 coupled to a single thick optical delivery fiber 113. Laser 112 could be any suitable laser such as gas laser, semiconductor laser, superluminescent laser diode, dye laser, Nd-YAG laser, Argon ion laser, or the like. Laser 112 may similarly comprise any array of lasers of the above-mentioned types, such as BAL or an array of BALs, laser diode known array-type laser, laser bar, stacked array, or a laser diode configuration type laser. In operation, thick optical delivery fiber 113 is brought in proximity to, or in contact with, or into, a biological tissue 114 to be treated. Biological tissue 114 may be an organ, a tumour or any other tissue. A second optical fiber 115 can be employed to collect light scattered by the tissue and deliver it to detection component 116.

Referring to FIG. 2, second configuration 120 is a PDT system whereby the employed laser is coupled through coupling optics to a thin optical delivery fiber. Configuration 120 comprises laser diode 122 coupled to coupling optics 123, 124, 125, 126. A portion of the beam of laser diode 122 is directed to feedback system 127 which is coupled to laser diode 122. Finally, laser diode 122 is coupled to thin optical delivery fiber 128. Thin optical delivery fiber 128 is brought in proximity to or in contact with or into the biological tissue 129 to be treated.

Biological tissue 129 is typically an organ, a tumour or any other tissue. An optical monitoring fiber or fibers 130 is employed to collect scattered light by biological tissue 129 and deliver it to a detection component 131.

It is desirable to be able to deliver power at the level that a BAL provides using more efficient and accurate approaches.

### SUMMARY

Devices and techniques are disclosed for delivering light from a plurality of single emitter lasers to a biological tissue and detecting light from a biological tissue with a plurality of detector components using multi-core optical delivery and detection fiber or fibers for minimally invasive treating and/or diagnosing conditions and/or diseases in an individual.

In one aspect, the device is used for photodynamic diagnosis and therapy and includes a first multi-core fiber to deliver a plurality of optical beams to light-irradiate a biological tissue, and a plurality of light sources each coupled to a different core of a first set of cores of the first multi-core fiber for generating the plurality of optical beams. The combined power level of the plurality of optical beams delivered to the biological tissue is above the power level threshold required to energize a photosensitizing drug.

In a further aspect, a plurality of photodetectors to detect optical beams scattered by the light-irradiated biological tissue.

In yet a further aspect, each of the plurality of detector components are coupled to each of a second set of cores of the first multi-core fiber.

In yet a further aspect, a second multi-core fiber, where each of the plurality of detector components are coupled to each of a first set of cores of the second multi-core fiber.

In another aspect, each of the plurality of light sources is a Single Emitter Laser (SEL).

In yet a further aspect, each of the plurality of lights sources and photodetectors are coupled to different cores of the first multi-core fiber through fibers.

In yet a further aspect where each of the plurality of light sources and photodetectors are coupled to different cores of the first multi-core fiber through free space coupling optics.

In yet a further aspect, each of the plurality of light sources and photodetectors are coupled to different cores of the first multi-core fiber through multi-core connectors

In another aspect , a set of half wave plates and a set of polarization beam combiners, where the plurality of light sources are grouped in groups of two, where a first light source of each group of two is coupled to a polarization beam combiner through a half wave plate, and a second light source of each group of two is coupled directly to the polarization beam combiner and where each polarization beam combiner is then coupled to a different core of the multi-core fiber in a manner that increase the amount of light energy delivered to the biological tissue.

In yet a further aspect, the half wave plates and the polarization beam combiners are at least one of free space and fiber components.

In yet a further aspect The device of claim 9, where the half wave plates and the polarization beam combiners are waveguide components integrated on a substrate.

In another aspect, the SELs are integrated on a first substrate and the plurality of photodetectors are integrated on a second substrate.

In yet a further aspect, each of the the plurality of SELs are coupled to cores of the multi-core fiber, and each of the plurality of photodetectors are coupled to the cores of the multi-core optical monitoring fiber, where the coupling is by butt-coupling or glue.

In yet a further aspect, the plurality of lasers and the plurality of photodetectors are integrated on a common substrate and coupled to the multi-core optical fiber through butt-coupling or glue.

In yet another further aspect, the size of each core of the multi-core fiber is selected to support either single-mode or multi-mode light propagation.

In another aspect, the distance between the cores and the power delivered through each core are selected to generate an arbitrary spatial irradiation profile to irradiate the biological tissue.

In yet another aspect, the therapeutic light originates from coherent addition of a plurality of single emitter lasers or from a single high power single emitter laser and the distance between cores in the multi-core fiber is such that light is guided as a single coherent mode to the biological tissue also achieving increased bend insensitivity and lower propagation loss.

In yet a further aspect, side illumination is performed by suitable post-processing of the delivery fiber at the distal end and selective lighting of the optical cores located at the periphery of the optical fiber.

In yet a further aspect, real-time variation of the light illuminated into the different cores leads to dynamic choice between distal end-illumination and side-illumination, where the individual optical power levels define the level of effect of the therapeutic light per direction of the biological tissue.

In yet a further exemplary embodiment, the tool is configured to uniformity of illumination of biological tissue on the basis of initially selecting initial illumination parameters of each of a set of outer cores of a multi-core fiber delivery, and then adjusting the illumination parameters on the basis of at least one of (i) detected feedback information on the absorption of the photosensitising agent per unit area for the case of PDT systems and (ii) visual inspection by a practitioner in the case of non-PDT procedures involving therapeutic light delivery.

### BRIEF DESCRIPTION OF THE DRAWINGS

FIGS. 1 and 2 illustrate two different types of conventional PDT configurations.

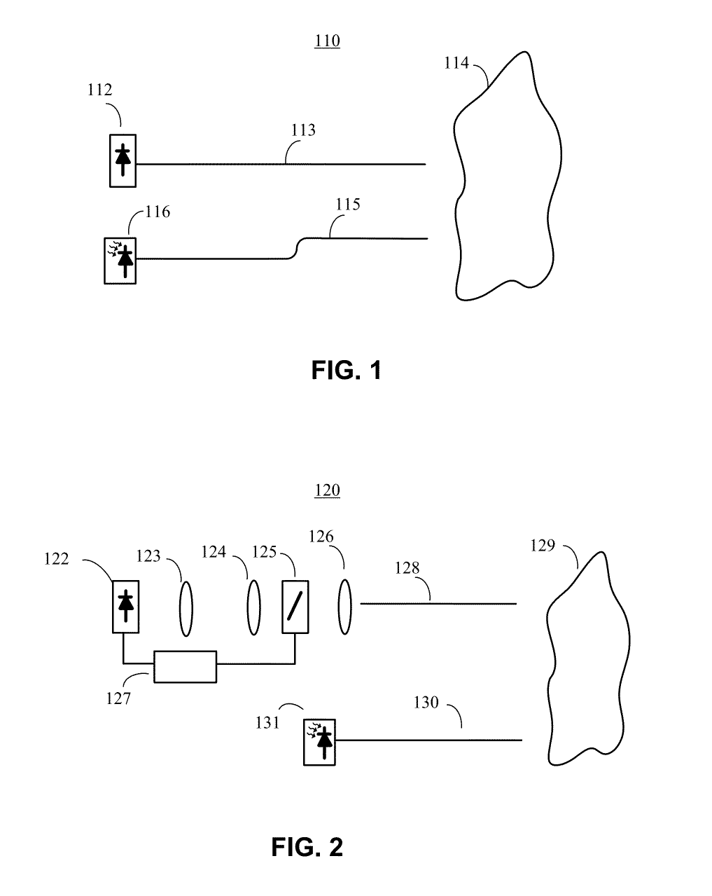

FIG. 3 is a cross-sectional view of a single-core, single mode optical fiber, a multi-core single mode optical fiber, a single core multi-mode optical fiber, and a multi-mode multi-core optical fiber.

FIG. 4 is a PDT laser shown with a plurality of lasers coupled through free-space optics to a multi-core optical delivery fiber according to an exemplary embodiment.

FIG. 5 is a PDT laser shown with a plurality of lasers coupled through a multi-core connector to a multi-core optical delivery fiber according to another exemplary embodiment.

FIG. 6 is a PDT laser shown with a plurality of lasers coupled to polarization beam combiners and then coupled through a multi-core connector (or through free space optics) to a multi-core optical delivery fiber according to a further exemplary embodiment.

FIG. 7 is a PDT system shown with a plurality of lasers coupled to a number of cores of a multi-core optical fiber and a plurality of detector components is coupled to a number of cores of the same multi-core fiber according to yet a further exemplary embodiment.

FIG. 8 is a PDT system shown with a plurality of lasers coupled to a multi-core optical delivery fiber and a plurality of detector components coupled to a different multi-core monitoring fiber according to another exemplary embodiment.

FIG. 9 is a PDT system shown with a plurality of lasers integrated on a substrate in a two-dimensional arrangement and then coupled to a multi-core optical delivery fiber and a plurality of detector components integrated on a substrate in a two-dimensional arrangement and then coupled to a multi-core optical monitoring fiber, in accordance with a further exemplary embodiment.

FIGS. 10 and 11 illustrate different PDT laser configurations with varying geometry of cores within the multi-core fibers to achieve different spatial light distribution in order to match desired characteristics of the irradiated biological tissue.

FIG. 12 illustrates a configuration implementation for side and/or end illumination in accordance with a further exemplary embodiment.

### DETAILED DESCRIPTION

The word “exemplary” is used herein to mean “serving as an example, instance, or illustration.” Any embodiment described herein as “exemplary” is not necessarily to be construed as preferred or advantageous over other embodiments.

The detailed description set forth below in connection with the appended drawings is intended as a description of exemplary embodiments of the present invention and is not intended to represent the only embodiments in which the present invention can be practiced. The “exemplary” embodiment should not necessarily be construed as preferred or advantageous over other exemplary embodiments. The detailed description includes specific details for the purpose of providing a thorough understanding of the exemplary embodiments of the invention. It will be apparent to those skilled in the art that the exemplary embodiments of the invention may be practiced without these specific details. In some instances, well known structures and devices are shown in block diagram form in order to avoid obscuring the novelty of the exemplary embodiments presented herein.

The proposed solution refers to devices and techniques for delivering power at the level that a BAL provides with the use of a single thin multi-core optical delivery fiber, thus enabling fast, and efficient treatment in a more minimally invasive way.

For specific interstitial treatments it is desirable to have a thinner delivery fiber with core diameter less than 400 um for minimally invasive diagnosis and treatment, delivering light with high spatial coherence. However, no laser system exists that can administer the Watt-level power required by PDT in a single fiber with core significantly smaller than 400 um and with the spatial coherence properties of Single Emitter Lasers (SELs).

In addition, SELs coupled to optical fibers with cores much smaller than 400 um can deliver power only in the range of mW range which, in itself, is not adequate for effective PDT.

It has been determined that it is desirable to have a light from a SEL launched into a single fiber with diameter less than 400 um for delivering Watt-level spatially coherent optical power required for PDT. It is also desirable to be able to control the spatial distribution of light along the wave-front in order match the tissue characteristics such as shape, depth, etc, to be irradiated.

Similarly, for accurate diagnosis or accurate treatment monitoring, it is further determined that it is desirable to collect the maximum possible light scattered by the biological tissue through the same delivery fiber or through a similarly thin optical monitoring fiber to detection components.

A solution is proposed herein whereby a single or a plurality of SELs are coupled to a single thin optical delivery fiber to enable fast and efficient treatment in a more minimally invasive way.

By controlling the power and distance of individual light beams emitted by SELs, spatial processing can be achieved and hence the average spatial light distribution can be changed as required.

It was further discovered that a plurality of detection components can be coupled to a single thin optical monitoring fiber to enable more accurate diagnosis and treatment monitoring. Multi-core fibers developed for increasing capacity in telecommunication networks are able to deliver high quality light from SELs, while also collecting scattered light to and from a biological tissue through thin optical fibers.

A PDT system that delivers high power light to a biological tissue and detects light scattered by a biological tissue through thin optical delivery fibers with multiple cores is proposed, as explained and described below in connection with the below referenced figures.

As shall be explained, the light energy delivered and collected to and from a biological tissue increases as the number of cores in the fiber increases. In addition, because the spatial distribution of delivered light can be easily adjusted by changing the geometry and placement of cores inside available multi-core fibers, irradiation of light distribution can be optimized to match desired tissue characteristics and treatment objectives.

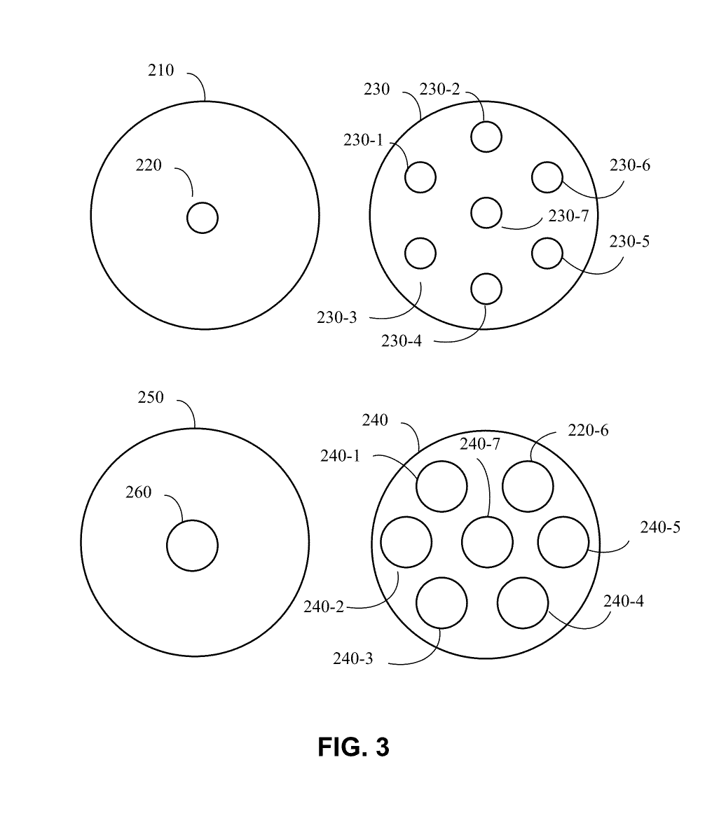

FIG. 3 is a cross-sectional view of a single-core, single mode optical fiber, a multi-core single mode optical fiber, a single core multi-mode optical fiber, and a multi-mode multi-core optical fiber.

The use of multi-core fibers is shown, in lieu of of having one core in the centre. Several cores dispalced in suitable distances. In this way, the total light energy delivered and the total scattered light energy collected is increased as the number of cores increases. In addition, light diffusion is more efficient in the case of multi-core fibers due to the positioning of cores closer to the cladding and due to their circular architecture.

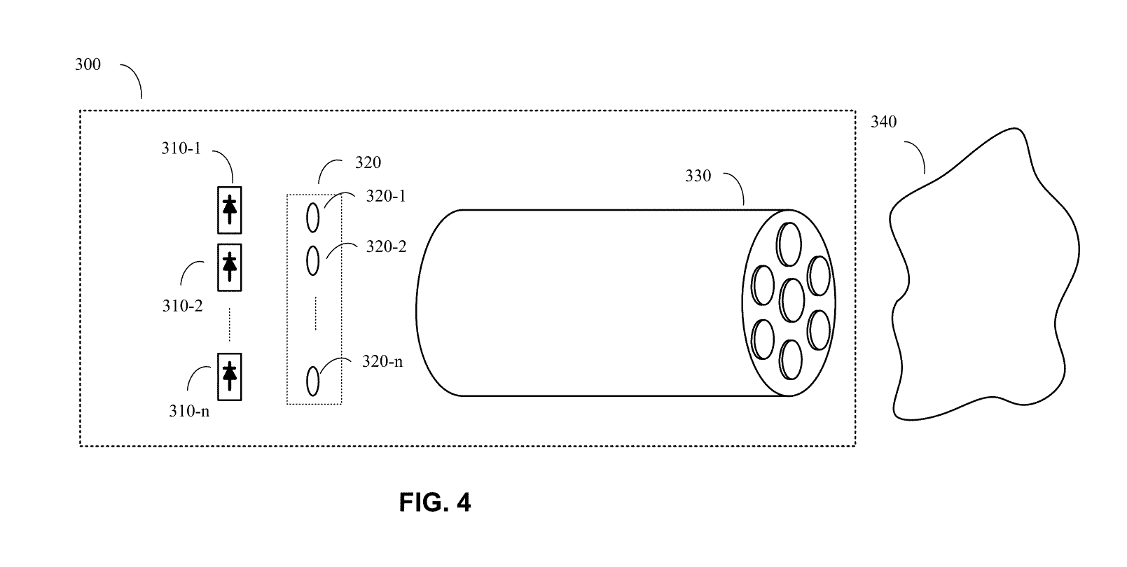

FIG. 4 is a PDT laser 300 shown with a plurality of lasers coupled through free-space optics to a multi-core optical delivery fiber according to an exemplary embodiment.

PDT laser 300 includes a plurality of lasers 310-1, 310-2, . . . , 310-n having arbitrary output powers and wavelengths. Lasers 310-1, 310-2 . . . , 310-n are coupled to different cores of multi-core optical delivery fiber 330 through free-space coupling optics 320-1, 320-2, . . . , 320-n. Coupling of lasers 310-1, 310-2, . . . , 310-n to different cores of the multi-core optical delivery fiber 330 can also be realized with optical fibers or through multi-core connector. Multi-core optical delivery fiber 330 is brought in proximity to or in contact with or into the biological tissue 340 to be treated. Biological tissue 340 maybe a skin surface, an organ, a tumour or any other tissue. Using this invention, the delivery of light from a plurality of SELs through the cores of a thin fiber that has multiple cores, results in a total power level, in the same power range as the total power delivered by a BAL coupled to a single thick fiber. Therefore, with the use of the plurality of SELs through a single multi-core fiber, it is possible to irradiate the biological tissue with a power level sufficient to energize the drug without the need to use a BAL that would require a single thick fiber to achieve the same level of power.

In the exemplary embodiment shown in FIG. 4, if seven SELs with 150 mW each were coupled to seven (7) cores, this would lead to a total power of more than 1 Watt through a thin optical fiber with diameter less than 250 μm and with the possibility to alter the spatial distribution on the wavefront by adjusting the power of each SEL. On the other hand, if a BAL would be used, the same amount of total power (1 Watt) would require a fiber with diameter at least 400 μm with poor spatial coherence properties along the wavefront.

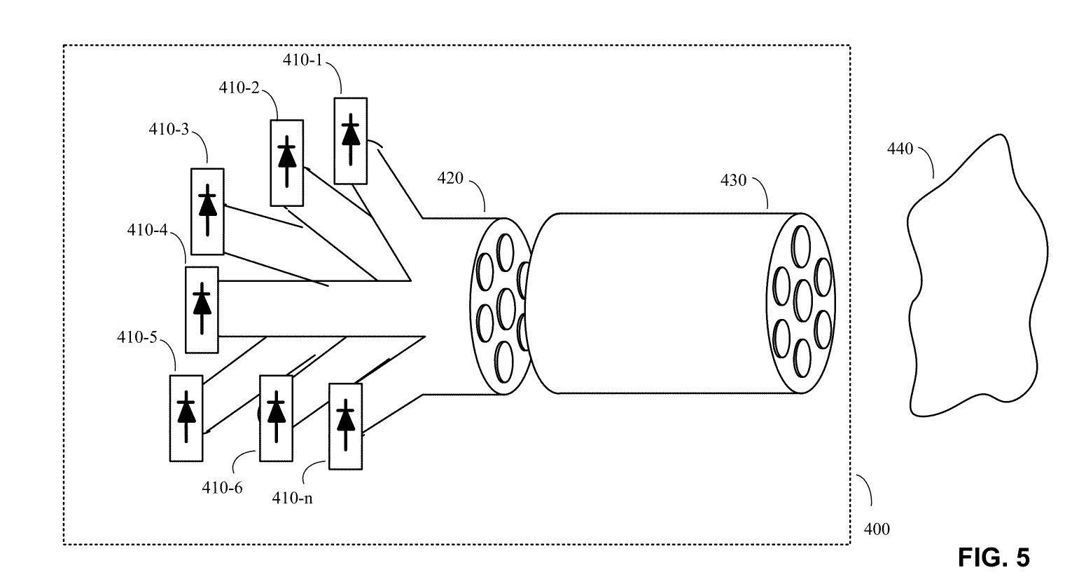

FIG. 5 is a PDT laser 400 shown with a plurality of lasers coupled through a multi-core connector to a multi-core optical delivery fiber according to another exemplary embodiment.

PDT laser 400 includes a plurality of lasers 410-1, 410-2, . . . , 410-n having arbitrary output powers and wavelengths. Lasers 410-1, 410-2, . . . , 410-n are coupled to different cores of multi-core optical delivery fiber 430 through multi-core connector 420. Coupling of lasers 410-1, 410-2, . . . , 410-n to different cores of the multi-core optical delivery fiber 430 can also be realized with optical fiber or with free-space coupling optics. Multi-core optical delivery fiber 430 is brought in proximity to or in contact with or into the biological tissue 440 to be treated. Biological tissue 440 may be a skin surface, an organ, a tumour or any other tissue.

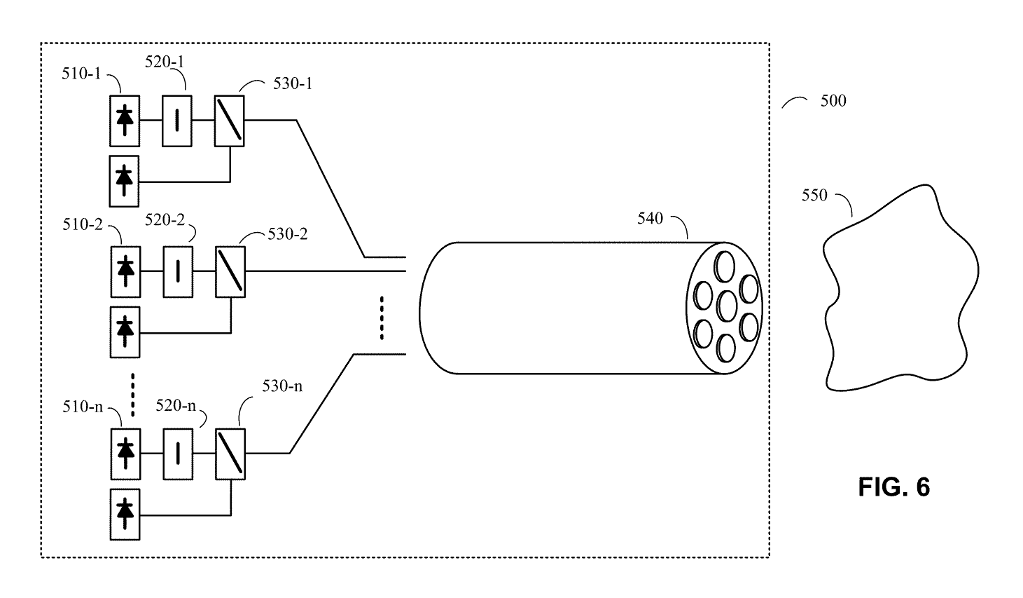

FIG. 6 is a PDT laser 500 shown with a plurality of lasers coupled to polarization beam combiners and then coupled through a multi-core connector (or through free space optics) to a multi-core optical delivery fiber according to a further exemplary embodiment.

PDT laser 500 includes a plurality of lasers 510-1, 510-2, . . . , 510-n having arbitrary output powers and wavelengths. Lasers 510-1, 510-2, . . . , 510-n are coupled in groups of two using half-wave plates 520-1, . . . , 520-n and polarization beam combiners 530-1, . . . , 530-n-1. The outputs of polarization beam combiners 530-1, . . . , 530-n are coupled to different cores of multi-core optical delivery fiber 540 through optical fiber.

Coupling of polarization beam combiner 530-1, . . . , 530-n outputs to different cores of the multi-core optical delivery fiber 540 can also be realized with free-space coupling optics or through multi-core connector. Multi-core optical delivery fiber 540 is brought in proximity to or in contact with or into the biological tissue 550 to be treated. Biological tissue 550 may be a skin surface, an organ, a tumour or any other tissue. Due to the polarization beam combination, PDT laser 500 can achieve higher light energy coupled to a biological tissue. Polarization components 520-1, 520-2, . . . , 520-n and 530-1, 530-2, . . . , 530-n can be free-space, fiber or waveguide components, i.e. integrated on a substrate.

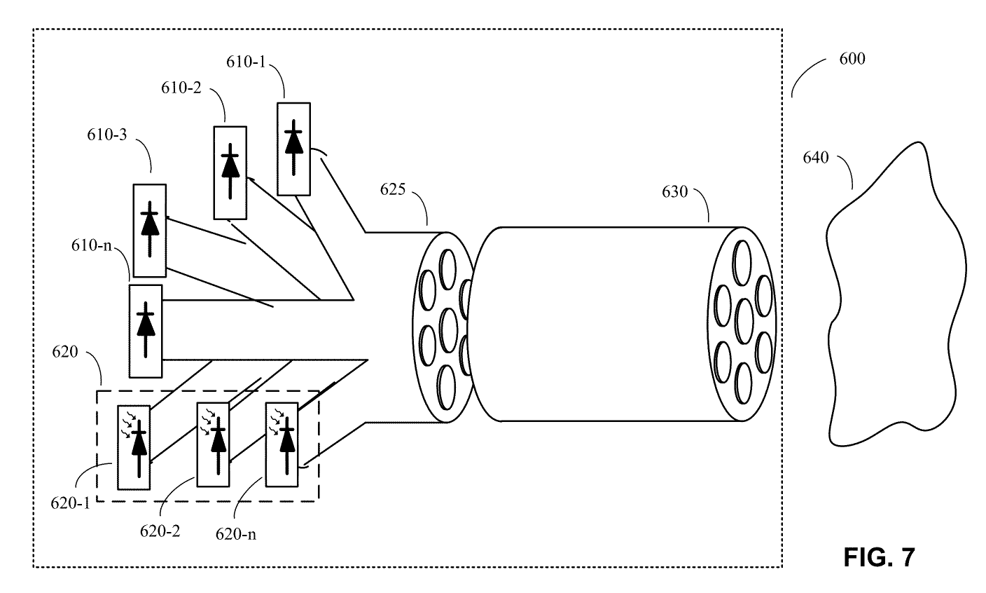

FIG. 7 is a PDT system 600 shown with a plurality of lasers coupled to a number of cores of a multi-core optical fiber and a plurality of detector components is coupled to a number of cores of the same multi-core fiber according to yet a further exemplary embodiment.

PDT system 600 includes a plurality of lasers 610-1, 610-2, 610-3, . . . , 610-n having arbitrary output powers and wavelengths. Lasers 610-1, 610-2, 610-3, . . . , 610-n are coupled to a number of cores of multi-core optical fiber 640 through optical fiber. Coupling of lasers 610-1, 610-2, 610-3, . . . , 610-n to the number of cores of multi-core optical fiber 630 can also be realized with free-space coupling optics or through multi-core connector. Multi-core optical fiber 630 is brought in proximity to or in contact with or into the biological tissue 640 to be treated. Biological tissue 640 may be a skin surface, an organ, a tumour or any other tissue.

PDT system 600 also includes a plurality of photodetectors 620-1, 620-2, . . . , 620-n. Photodetectors 620-1, 620-2, . . . , 620-n are coupled to a number of cores of multi-core optical fiber 630 by similar means as lasers 610-1, 610-2, . . . , 610-n. Thus multi-core fiber 630 can be used simultaneously as delivery fiber delivering light to tissue 640 and as monitoring fiber collecting light scattered by tissue 640.

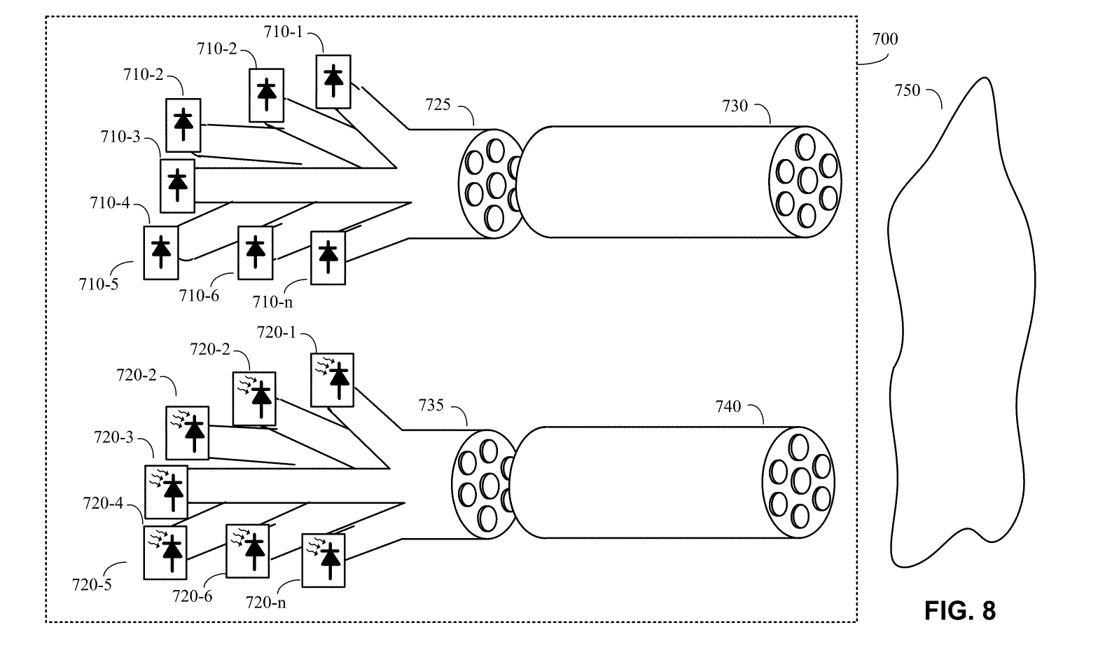

FIG. 8 is a PDT system 700 shown with a plurality of lasers coupled to a multi-core optical delivery fiber and a plurality of detector components coupled to a different multi-core monitoring fiber according to another exemplary embodiment.

PDT system (700) includes a plurality of lasers 710-1, 710-2, . . . , 710-n having arbitrary output powers and wavelengths. Lasers 710-1, 710-2, . . . , 710-n are coupled to different cores of multi-core optical delivery fiber 730 through optical fiber. Coupling of lasers 710-1, 710-2, . . . , 710-n to different cores of the multi-core optical delivery fiber (730) can also be realized with free-space coupling optics or through multi-core connector. Multi-core optical delivery fiber 730 is brought in proximity to or in contact with or into the biological tissue 750 to be treated. Biological tissue 750 maybe a skin surface, an organ, a tumour or any other tissue. PDT system 700 also includes a plurality of photodetectors 720-1, 720-2, . . . , 720-n. Photodetectors 720-1, 720-2, . . . , 720-n are coupled to different cores of multi-core optical monitoring fiber 740 by similar means as lasers 710-1, 710-2, . . . , 710-n are coupled to different cores of multi-core delivery fiber 730. Multi-core optical monitoring fiber 740 is brought in proximity to or in contact with or into the biological tissue 750 to be treated.

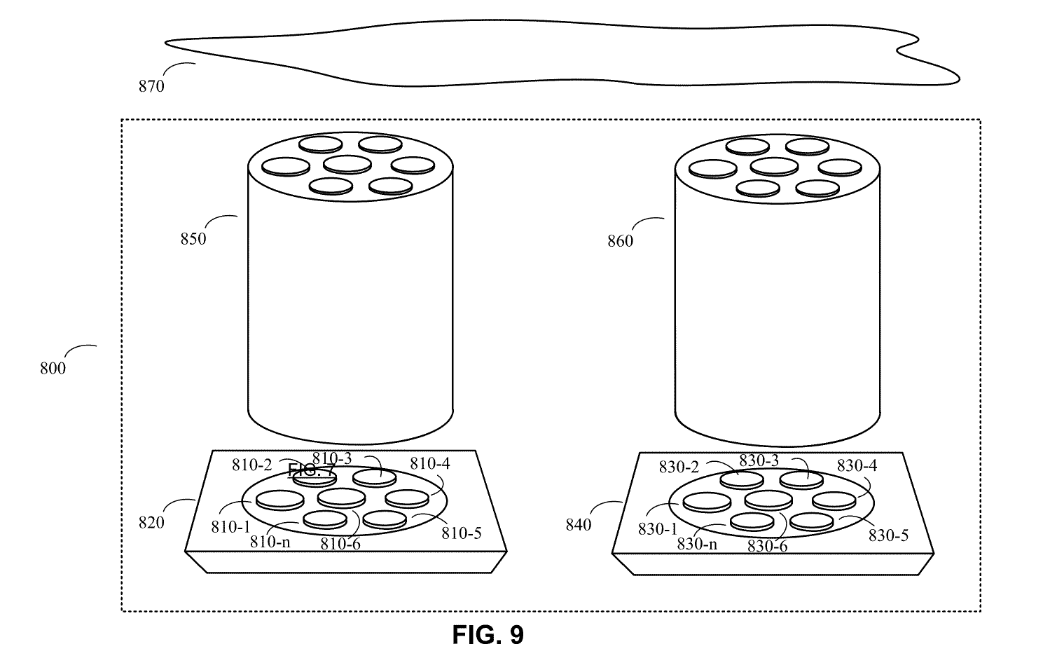

FIG. 9 is a PDT system 800 shown with a plurality of lasers integrated on a substrate in a two-dimensional arrangement and then coupled to a multi-core optical delivery fiber and a plurality of detector components integrated on a substrate in a two-dimensional arrangement and then coupled to a multi-core optical monitoring fiber, in accordance with a further exemplary embodiment.

PDT system 800 includes a plurality of lasers 810-1, 810-2, . . . , 810-n having arbitrary output powers and wavelengths. Lasers 810-1, 810-2, . . . , 810-n are integrated on a common substrate 820. The geometrical arrangement of lasers 810-1, 810-2, . . . , 810-n on substrate 820 is such that the lasers are pitch-matched to the cores of multi-core optical delivery fiber 850. Coupling of lasers 810-1, 810-2, . . . , 810-n to different cores of the multi-core optical delivery fiber 830 can be realized with butt-coupling or gluing techniques.

To enable such coupling lasers 810-1, 810-2, . . . , 810-n should be typically vertically emitting components such as semiconductor VCSELs. Multi-core optical delivery fiber 850 is brought in proximity to or in contact with or into the biological tissue 870 to be treated. Biological tissue 860 maybe an organ, a tumour or any other tissue. PDT system 800 also includes a plurality of photodetectors 830-1, 830-2, . . . , 830-n. Photodetectors 830-1, 830-2, . . . , 830-n are integrated on a common substrate 840. The geometrical arrangement of photodetectors 830-1, 830-2, . . . , 830-n on substrate 840 is such that the photodetectors are pitch-matched to the cores of multi-core optical monitoring fiber 860.

Multi-core optical monitoring fiber 860 is brought in proximity to or in contact with or into the biological tissue 870 to be treated. Biological tissue 870 may be a skin surface, an organ, a tumour or any other tissue. One skilled in the art may appreciate that photodetectors 830-1, 830-2, . . . , 830-n can be integrated on a common substrate with lasers 810-1, 810-2, . . . , 810-n and then one common multi-core fiber can be used for light delivery and monitoring purposes.

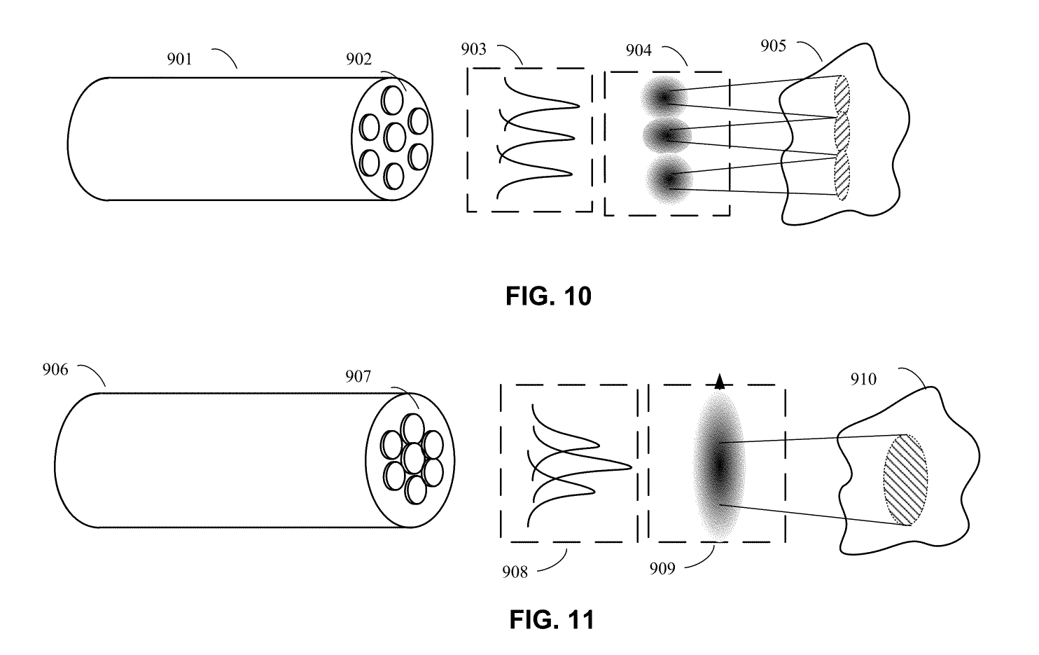

FIGS. 10 and 11 illustrate different PDT laser configurations with varying geometry of cores within the multi-core fibers to achieve different spatial light distribution in order to match desired characteristics of the irradiated biological tissue. More specifically, FIGS. 10 and 11 illustrate (i) where strong or weak coupling between cores is induced and (ii) how optimization of irradiation can be achieved when changing the core diameter, pitch, geometry and optical power within each core. In this regard, the multi-core fibers illustrated in FIGS. 10 and 11 can be illuminated with any type of horizontally or vertically emitting SEL or SELs which are not shown for simplicity.

Referring to FIG. 10, here the PDT system shown is characterized by weak or no coupling of light between cores 902 within multi-core fiber 901. The weak coupling of the cores 902 results in the SEL spatial distribution 90) where the light beams have equal power maxima. Item 904 is the cross-section of the SEL spatial distribution 903. Multi-core optical delivery fiber 901 is brought in proximity to or in contact with or into the biological tissue 905 to be treated which is irradiated with the light pattern shown in the figure. Biological tissue 905 maybe a skin surface, an organ, a tumour or any other tissue.

Referring to FIG. 11, the PDT system here involves strong coupling of light between cores 907 within multi-core fiber 906. The strong coupling of the light within the cores 907 results in the SEL spatial distribution 908 where the light beams have unequal power maxima, with a strong lobe in the middle of the combined beam and a number of weak lobes on the beam edges. Item 909 represents the cross-section of the SEL spatial distribution 908. Multi-core optical delivery fiber 906 is brought in proximity to or in contact with or into the biological tissue 910 to be treated which is irradiated with the light pattern shown in the figure. Biological tissue 910 may be a skin surface, an organ, a tumour or any other tissue.

FIGS. 10 and 11 show cases of either strong or weak coupling between cores being induced. However, it is to be appreciated that the exemplary embodiments can also be implemented with multi-core fibers that have different core numbers, with arbitrary core-to-core distances and geometries for allowing custom spatial distribution of light irradiating biological tissues.

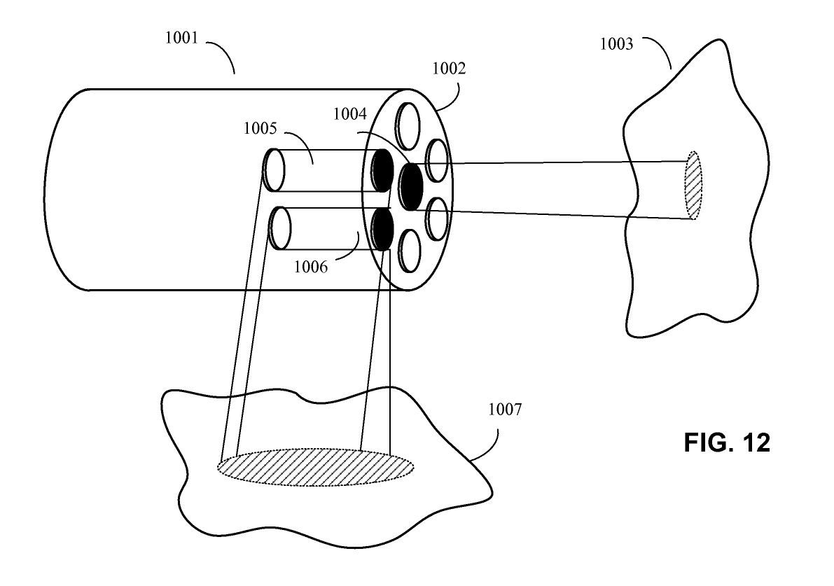

FIG. 12 illustrates a configuration implementation for side and/or end illumination in accordance with a further exemplary embodiment. Here, multi-core light delivery fiber 1001 is used to guide light into the different cores of the fiber 1002. According to a proposed principle of operation, light illuminates only central core 1004 thus achieving strong end-illuminated apparatus and hence illuminating the biological tissue 1003.

Alternatively, light may selectively illuminate a selection of the outer cores of the multi-core fiber (cores 1005 and 1006), thus achieving strong side illumination of the biological tissue 1007.

The proposed solution that achieves treatment efficiency, treatment duration and treatment monitoring improvements resulting substantially from the utilization of multi-core fibers with optimized core geometry that couple a plurality of lasers and detector components.

The number of cores, the distance between them and the launched power in each core are the three parameters that enable the manipulation of the spatial distribution of light and hence allows for altering the spatial coherence properties of the resultant light beam.

In a further exemplary embodiment not shown, a software tool may be employed which receives various parameters, including spatial and thickness characteristics, stage of progression, type of tumour, and many other related properties and characteristics, and automatically selects an optimum configuration of lasers to be active and irradiated, and the power level, interval and increment, whether continuous or pulsed, or other configuration type parameters which a skilled technician would take into account in providing optimum treatment.

The tool, as contemplated, would configure the lasers and intensity in manner which would not be otherwise feasible or practical for a physican, surgeon or technician of conventional equipment. The efficiency and accuracy of use of the proposed lasers described above would be further enhanced by such a tool.

The tool may further employ visual aids which may further assist the surgeon in application of the lasers during treatment using unused light sources as a return path for visual (or even audio) from the region under treatement.

In yet a further exemplary embodiment, the tool is configured to uniformity of illumination of biological tissue on the basis of initially selecting initial illumination parameters of each of a set of outer cores of a multi-core fiber delivery, and then adjusting the illumination parameters on the basis of at least one of (i) detected feedback information on the absorption of the photosensitising agent per unit area for the case of PDT systems and (ii) visual inspection by a practitioner in the case of non-PDT procedures involving therapeutic light delivery.

Minimally invasive treatment is enabled by the fact that the multi-core fibers are thin fibers, having fiber radius of 250 um or less. Moreover, the core physical separation and individual laser power levels in each core can be designed so as to form a spatially coherent light beam with optical power that can be used for PDT.

In an exemplary embodiment, the PDT system involves a plurality of SELs coupled to a single mode multi-core optical delivery fiber. Laser to multi-core fiber coupling can be made through fiber, free-space optics, integrated waveguides, multi-core connectors or directly with butt-coupling of the multi-core fiber to the lasers. The plurality of lasers may comprise any suitable laser such as gas laser, semiconductor laser, superluminescent laser diode, dye laser, Nd-YAG laser, Argon ion laser etc or any combination thereof.

The plurality of lasers may comprise any array of lasers of the above-mentioned types, such as broad area laser or an array of broad area lasers, laser diode array, laser bar or stacked array, laser diode, or the like.

Typically, lasers operate at wavelengths ranging from about 450 nm to about 900 nm with 630 nm being most typical for photosensitizer agent excitation. The wide range of supported wavelengths implies that the PDT system can be used with similar efficiency to other medical treatment process such as wound healing.

Lasers can be also operated in continuous wave (CW) mode or be modulated for pulsed mode operation. In addition, lasers can be set to operate independently or in phase locked mode. The number of cores and their geometrical arrangement in the multi-core optical fiber is directly related to the requirements of the diagnostic or treatment modality in which they are to be used.

Typical single mode multi-core fibers involve seven cores arranged in a hexagonal arrangement for producing seven independent light beams with weak coupling between the signals.

For illustration purposes, this arrangement is employed in the figures that follow to describe a number of alternate exemplary embodiments of the present invention. One skilled in the art would readily appreciate, however, that any multi-core fiber with any number of cores may be used in substitution provided that the total combined power of the independent light beams is above a predetermined desired power threshold.

The distance between cores is adjusted and made smaller in order to increase the coupling between light travelling into different cores. These can also be combined, each having a different irradiation intensity (power level) to contemporaneously irradiate a tissue to match the thickness and geometry (shape) of the irradiated upon tissue or other object.

Single mode multi-core optical fibers may be both passive or active and can be of any suitable material enabling light guiding such as silica, plastic, photonic crystal, polymer, etc.

Single mode multi-core fibers may also function as diffusing fibers and/or can be made to bear any written component such as Bragg grating. Moreover, through post-processing of the delivery fiber, side illumination of the multi-core fiber can be also achieved by conventional techniques.

In addition, specific focusing components (such as ball lenses) can be fitted to the multicore fiber distal end for light processing purposes such as focusing or diffusing. One skilled in the art may appreciate that the present invention can be also implemented in a similar way using multi-mode multi-core fibers instead of single-mode multi-core fibers with the proper selection of the corresponding multi-mode components in case the treatment modality requires multi-mode beams.

The PDT system also involves a plurality of detector components coupled to the same optical multi-core delivery fiber or to a different optical multi-core monitoring fiber. The plurality of detector components may comprise any suitable photodetector such as semiconductor photodetectors. Coupling can be made through free-space optics, integrated waveguides, multi-core connectors or directly with butt-coupling of the multi-core fiber to the detector components.

For applications extending beyond PDT systems, where therapeutic light is used as the means of direct treatment, the disclosed invention can provide user-controlled illumination patterns, selection of illumination directions as well as accurate real-time control of light intensity by varying the distribution of light within the cores of the delivery fiber. \As an example of the unique properties of the invention, in the case where real-time selection between end- and side-illumination is required real-time during operation, this can be achieved by injecting more light in the central core and less light into the circular cores and vice-versa.

Moreover, in the case where selective side light illumination of therapeutic light is required, this is achieved by controlling the optical power injected into the different circular cores. In the specific application of vane coagulation, the precise control of light illumination direction and intensity leads to more efficient and uniform treatment of varicose veins.

Depending on the optical power injected into each core, combination of side- and end-illumination can be achieved if required by the application to which the invention is used for.

The aforementioned figure describe the use of single multi-core optical delivery and a single multi-core monitoring fiber, or alternatively, simultaneous use of a common multi-core optical fiber for delivery and monitoring. One skilled in the art should however readily appreciate that the number of multi-core fibers used for light delivery and monitoring can be increased depending on the requirements of the diagnostic or treatment modality without departing from the scope and purpose of the invention.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A device for photodynamic diagnosis and therapy comprising:
a first multi-core fiber to deliver a plurality of optical beams to light-irradiate a biological tissue; and
a plurality of light sources each coupled to a different core of a first set of cores of the first multi-core fiber for generating the plurality of optical beams, where the combined power level of the plurality of optical beams delivered to the biological tissue is above the power level threshold required to energize a photosensitizing drug.

2. The device of claim 1, further comprising a plurality of photodetectors to detect optical beams scattered by the light-irradiated biological tissue.

3. The device of claim 2, where each of the plurality of detector components are coupled to each of a second set of cores of the first multi-core fiber.

4. The device of claim 2, further comprising a second multi-core fiber, where each of the plurality of detector components are coupled to each of a first set of cores of the second multi-core fiber.

5. The device of claim 2, where each of the plurality of light sources is a Single Emitter Laser (SEL).

6. The device of claim 2, where each of the plurality of lights sources and photodetectors are coupled to different cores of the first multi-core fiber through fibers.

7. The device of claim 2, where each of the plurality of light sources and photodetectors are coupled to different cores of the first multi-core fiber through free space coupling optics.

8. The device of claim 2, where each of the plurality of light sources and photodetectors are coupled to different cores of the first multi-core fiber through multi-core connectors.

9. The device of claim 2, further comprising a set of half wave plates and a set of polarization beam combiners, where the plurality of light sources are grouped in groups of two, where a first light source of each group of two is coupled to a polarization beam combiner through a half wave plate, and a second light source of each group of two is coupled directly to the polarization beam combiner and where each polarization beam combiner is then coupled to a different core of the multi-core fiber in a manner that increase the amount of light energy delivered to the biological tissue.

10. The device of claim 9, where the half wave plates and the polarization beam combiners are at least one of free space and fiber components.

11. The device of claim 9, where the half wave plates and the polarization beam combiners are waveguide components integrated on a substrate.

12. The device of claim 5, where the SELs are integrated on a first substrate and the plurality of photodetectors are integrated on a second substrate.

13. The device of claim 12, where each of the the plurality of SELs are coupled to cores of the multi-core fiber, and each of the plurality of photodetectors are coupled to the cores of the multi-core optical monitoring fiber, where the coupling is by butt-coupling or glue.

14. The device of claim 13, where the plurality of lasers and the plurality of photodetectors are integrated on a common substrate and coupled to the multi-core optical fiber through butt-coupling or glue.

15. The device of claim 1, where the size of each core of the multi-core fiber is selected to support either single-mode or multi-mode light propagation.

16. The device of claim 1, where the distance between the cores and the power delivered through each core are selected to generate an arbitrary spatial irradiation profile to irradiate the biological tissue.

17. The device of claim 1, where the therapeutic light originates from coherent addition of a plurality of single emitter lasers or from a single high power single emitter laser and the distance between cores in the multi-core fiber is such that light is guided as a single coherent mode to the biological tissue also achieving increased bend insensitivity and lower propagation loss.

18. The device of claim 1, where side illumination is performed by suitable post-processing of the delivery fiber at the distal end and selective lighting of the optical cores located at the periphery of the optical fiber.

19. The device of claim 1, where real-time variation of the light illuminated into the different cores leads to dynamic choice between distal end-illumination and side-illumination, where the individual optical power levels define the level of effect of the therapeutic light per direction of the biological tissue.

20. A method of optimizing uniformity of illumination of biological tissue comprising:
selecting illumination parameters of each of a set of outer cores of a multi-core fiber delivery; and
adjusting the illumination parameters on the basis of at least one of (i) detected feedback information on the absorption of the photosensitising agent per unit area for the case of PDT systems and (ii) visual inspection by a practitioner in the case of non-PDT procedures involving therapeutic light delivery.
